(19) United States
(12) Patent Application Publication
(10) Pub. No.: US 2024/0343572 A1
(43) Pub. Date: Oct. 17, 2024

(54) TREATMENT APPARATUS USING REACTIVE OXYGEN AND TREATMENT METHOD USING REACTIVE OXYGEN

(71) Applicant: CANON KABUSHIKI KAISHA, Tokyo (JP)

(72) Inventors: MASATSUGU HONGO, Shizuoka (JP); ATSUSHI UEMATSU, Shizuoka (JP); YUICHI KIKUCHI, Shizuoka (JP); TAKUMI FURUKAWA, Shizuoka (JP); KAZUHIRO YAMAUCHI, Shizuoka (JP); NAOYA ASANUMA, Shizuoka (JP); JUNKO HIRATA, Shizuoka (JP); TORU OGUMA, Shizuoka (JP)

(21) Appl. No.: 18/751,480

(22) Filed: Jun. 24, 2024

Related U.S. Application Data
(63) Continuation of application No. PCT/JP2022/048047, filed on Dec. 26, 2022.

(30) Foreign Application Priority Data
Dec. 28, 2021 (JP) 2021-215339

(51) Int. Cl. C01B 13/02; A61L 2/20; H05H 1/24

(52) U.S. Cl. CPC C01B 13/0203; A61L 2/20; H05H 1/2418; H05H 1/2425; A61L 2202/11; H05H 2245/36; H05H 2245/40

(57) ABSTRACT

A treatment apparatus and a treatment method using reactive oxygen, wherein, the treatment apparatus comprising: the reactive oxygen supply device comprises a plasma actuator and an ozone decomposing device inside a housing having at least one opening portion, the plasma actuator comprises a first electrode and a second electrode across a dielectric material and generates an induced flow containing ozone when a voltage is applied between the both electrodes, the plasma actuator and the ozone decomposing device are arranged such that the induced flow flows out from the opening portion and the induced flow is supplied to a surface of the object which is conveyed by the conveying means, and an outflow direction vector of the induced flow has a vector component x which is parallel to and oriented in the same direction as specific direction A.

### CROSS-REFERENCE TO RELATED APPLICATION

This is a continuation of International Application No. PCT/JP2022/048047, filed on Dec. 26, 2022, and designated the U.S., and claims priority from Japanese Patent Application No. 2021-215339 filed on Dec. 28, 2021, the entire contents of which are incorporated herein by reference.

### BACKGROUND OF THE INVENTION

### Field of the Invention

The present disclosure is directed to a treatment apparatus using reactive oxygen and a method of treating an object to be treated using reactive oxygen.

### Background Art

PTL 1 discloses a surface treatment apparatus capable of increasing a treatment effect due to ultraviolet rays and ozone. The surface treatment apparatus is described as in which an ultraviolet generating lamp is equipped inside a box-like apparatus main body, a plate body with high ultraviolet transmittance is provided on a corresponding surface of the ultraviolet lamp, an interior of the apparatus main body is set to an atmosphere with high ultraviolet transmittance, and an ozone treatment space is provided below the plate body. Moreover, It is described that the surface treatment apparatus sets the interior of the apparatus main body to an atmosphere with high ultraviolet transmittance by supplying nitrogen gas or an inert gas to the interior of the apparatus main body with turning on the ultraviolet generating lamp, or vacuumizing the interior of the apparatus main body, and further supplies ozone through an ozone supply port and, for example, when treating a sample conveyed by a conveyor, the sample is subjected to ultraviolet treatment by ultraviolet rays of 185 nm and 254 nm which are generated by the ultraviolet generating lamp and which are efficiently transmitted through the plate body and the sample is treated with ozone in the ozone treatment space. Moreover, it is described that since the surface treatment apparatus is provided with the ozone treatment space below the plate body, in the ozone treatment space, ozone is oxidatively decomposed by the ultraviolet rays having been transmitted through the plate body and a surface of the sample is effectively sterilized, and needless oxidative decomposition of ozone can be prevented.

The present inventors examined the surface treatment apparatus according to PTL 1 and found that a treatment effect with respect to a sample moving inside the ozone treatment space was limited.

At least one aspect of the present disclosure is directed to provide a treatment apparatus using reactive oxygen and a treatment method using reactive oxygen capable of more effectively treating a moving object to be treated.

### CITATION LIST

### Patent Literature

PTL 1: Japanese Patent Application Publication No. H01-025866

### Non Patent Literature

NPL 1: “Magnetic Field Effect on the Photocatalytic Reaction with TiO₂ Semiconductor Film” (Bulletin of the Society of Photography and Imaging of Japan, 2006, 69, 4, 271-275).

### SUMMARY OF THE INVENTION

According to at least one aspect of the present disclosure, there is provided that a treatment apparatus using reactive oxygen, comprising:
a reactive oxygen supply device and conveying means capable of conveying an object to be treated with reactive oxygen in at least a direction A, wherein
the reactive oxygen supply device comprises a plasma actuator and an ozone decomposing device inside a housing having at least one opening portion,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge from the first electrode to the second electrode and cause an induced flow containing ozone to be blown out from the first electrode in a first direction, which is one direction along the surface of the dielectric material,
the ozone decomposing device generates reactive oxygen in the induced flow by decomposing the ozone contained in the induced flow and the induced flow becomes an induced flow containing reactive oxygen,
the plasma actuator and the ozone decomposing device are arranged such that the induced flow containing the reactive oxygen flows out from the opening portion to the outside of the housing,
an outflow direction vector of the induced flow containing the reactive oxygen flowed out from the opening portion to the outside of the housing has a vector component x which is parallel to and oriented in the same direction as direction A, and
the reactive oxygen supply device and the conveying means are arranged such that the induced flow containing the reactive oxygen which flows out from the opening portion to the outside of the housing is supplied to a surface of the object to be treated which is conveyed by the conveying means.

Moreover, according to at least one aspect of the present disclosure, there is provided that a treatment method of treating an object to be treated using reactive oxygen, comprising:
a step of preparing a reactive oxygen supply device and conveying means capable of conveying the object to be treated with reactive oxygen in at least a direction A, wherein
the reactive oxygen supply device comprises a plasma actuator and an ozone decomposing device inside a housing having at least one opening portion,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge from the first electrode to the second electrode and cause an induced flow containing ozone to be blown out from the first electrode in a first direction, which is one direction along the surface of the dielectric material,
the ozone decomposing device generates reactive oxygen in the induced flow by decomposing the ozone contained in the induced flow and the induced flow becomes an induced flow containing reactive oxygen,
the plasma actuator and the ozone decomposing device are arranged such that the induced flow containing the reactive oxygen flows out from the opening portion to the outside of the housing,
the treatment method further comprises a step of causing the induced flow containing the reactive oxygen to flow out from the opening portion and supplying the induced flow containing the reactive oxygen to the object to be treated, which is moved in the direction A, and
an outflow direction vector of the induced flow containing the reactive oxygen flowed out from the opening portion has a vector component x which is parallel to and oriented in the same direction as the direction A.

Further features of the present invention will become apparent from the following description of exemplary embodiments with reference to the attached drawings.

### BRIEF DESCRIPTION OF THE DRAWINGS

FIG. 1A is a schematic cross-sectional view illustrating a configuration of a treatment device using reactive oxygen according to one aspect of the present disclosure, and FIG. 1B is a schematic cross-sectional view illustrating a configuration of the treatment device according to another aspect of the present disclosure.

FIG. 2A is a schematic sectional view showing a configuration of a plasma actuator according to the aspect of the present disclosure, and FIG. 2B is a diagram when the plasma actuator is viewed through from a side of one surface of the dielectric material.

FIG. 3 is an explanatory diagram of the plasma actuator according to the aspect of the present disclosure.

FIG. 4 is a dimension explanatory diagram of a reactive oxygen supply device according to the aspect of the present disclosure.

FIG. 5A is an explanatory diagram illustrating a plan view obtained by viewing from the first electrode side, and FIG. 5B is a perspective view obtained by viewing the plasma actuator from the first electrode side thereof.

FIG. 6 is a schematic view showing a relationship between an outflow direction vector of an induced flow and a direction A.

### DESCRIPTION OF THE EMBODIMENTS

In the present disclosure, the expression of “from XX to YY” or “XX to YY” indicating a numerical range means a numerical range including a lower limit and an upper limit which are end points, unless otherwise specified. Also, when a numerical range is described in a stepwise manner, the upper and lower limits of each numerical range can be arbitrarily combined. In addition, in the present disclosure, for example, descriptions such as “at least one selected from the group consisting of XX, YY and ZZ” mean any of XX, YY, ZZ, the combination of XX and YY, the combination of XX and ZZ, the combination of YY and ZZ, and the combination of XX, YY, and ZZ.

In the present disclosure, “treatment” of an object to be treated using reactive oxygen includes every treatment that can be achieved by reactive oxygen such as surface modification (hydrophilization treatment), sterilization, deodorization, and bleaching of a surface to be treated of the object to be treated using reactive oxygen.

Further, “bacteria” as an object to be subjected to the “sterilization” according to the present disclosure refers to microorganisms, and the microorganisms include fungi, bacteria, unicellular algae, viruses, protozoa, and the like, as well as animal or plant cells (including stem cells, dedifferentiated cells, and differentiated cells), tissue cultures, fused cells obtained by genetic engineering (including hybridomas), dedifferentiated cells, and transformants (microorganisms). Examples of the viruses include norovirus, rotavirus, influenza virus, adenovirus, coronavirus, measles virus, rubella virus, hepatitis virus, herpes virus, HIV virus, and the like. Examples of the bacteria include Staphylococcus, Escherichia coli, Salmonella, Pseudomonas aeruginosa, Vibrio cholerae, Shigella, Anthrax, Mycobacterium tuberculosis, Clostridium botulinum, Tetanus, Streptococcus, and the like. Furthermore, examples of the fungi include Trichophyton, Aspergillus, Candida, and the like. Accordingly, the “sterilization” according to the present disclosure includes even virus inactivation.

In addition, the reactive oxygen in the present disclosure includes, for example, a free radical caused by decomposition of ozone (O₃), such as superoxide (·O₂⁻) or hydroxy radical (·OH).

Referring to the drawings, a mode for carrying out this disclosure will specifically be described below by way of example. Note that dimensions, materials, shapes, relative positioning, and the like of components described in the mode are to be appropriately changed depending on configurations of members to which the disclosure is applied and various conditions. In other words, it is not intended to limit the scope of this disclosure to the following modes. Furthermore, in the following description, components having the same functions are denoted by the same numerals in the drawings, and a description thereof may be omitted.

The present inventors infer the reason why the treatment effect with respect to a sample moving inside the ozone treatment space is limited in the surface treatment apparatus according to Patent Literature 1 as follows.

In the ozone treatment space of the surface treatment apparatus according to Patent Literature 1, it is considered that ozone present in the ozone treatment space is decomposed by ultraviolet rays and reactive oxygen is generated. In this case, reactive oxygen collectively refers to reactive oxygen species with high reactivity such as superoxide anion radical (O₂⁻) and hydroxyl radical (OH) which are capable of instantaneously oxidatively decomposing bacteria and viruses due to intrinsic high reactivity. In addition, a sample passes through the ozone treatment space in which reactive oxygen is generated while being conveyed by a conveyor. In the process, treatment such as sterilization of the sample is performed. However, reactive oxygen is extremely unstable, with extremely short half-lives such as a half-life of O₂⁻ being 10⁻⁶ seconds and a half-life of OH being 10⁻⁹ seconds, and quickly converted into oxygen or water which are stable. Therefore, when the sample is conveyed in the ozone treatment space, it is considered that a turbulence of air flow is created inside the ozone treatment space due to the conveyance of the sample and a substantial amount of the reactive oxygen inside the ozone treatment space collides with a wall or the like in the treatment space and changes to oxygen or water. In other words, a ratio used to treat the sample of the reactive oxygen generated in the ozone treatment space is low. Therefore, it is considered that, in the surface treatment apparatus according to Patent Literature 1, the treatment effect with respect to a sample moving inside the ozone treatment space is limited.

Based on the consideration described above, it was recognized that in order to treat a moving object to be treated more effectively using reactive oxygen with a short lifespan, the object to be treated or a surface to be treated must be more actively placed in a reactive oxygen atmosphere. And, based on such recognition, as a result of the examination by inventors, it was found that treatment apparatuses according to the various aspects to be described below can cause reactive oxygen to reliably reach a moving object to be treated while maintaining treatment capability of the reactive oxygen. And, as a result, it was further discovered that the object to be treated can be more actively placed in a reactive oxygen atmosphere and treatment efficiency of the object to be treated can be dramatically improved.

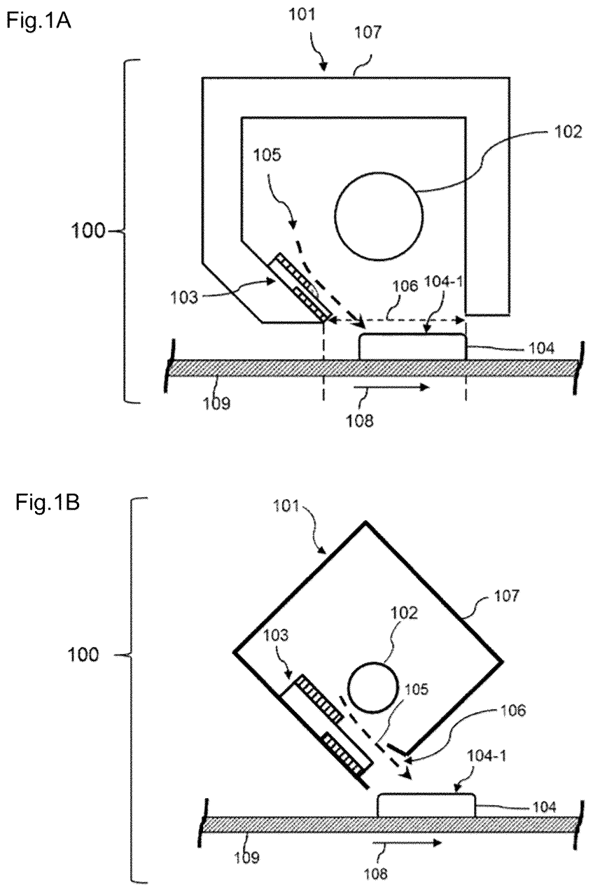

Hereinafter, a treatment apparatus 100 according to an aspect of the present disclosure will be described using FIG. 1A. The treatment apparatus 100 comprises a reactive oxygen supply device 101 and conveying means 109 capable of conveying an object to be treated 104 in a direction (a direction A) of an arrow 108.

The reactive oxygen supply device 101 according to the embodiment of the present disclosure comprises, inside a housing 107 having at least one opening portion 106, a UV light source 102 serving as the ozone decomposing device 102 and a plasma actuator 103.

The UV light source serving 102 as the ozone decomposing device irradiates induced flows 105 with UV light to generate reactive oxygen in the induced flows 105.

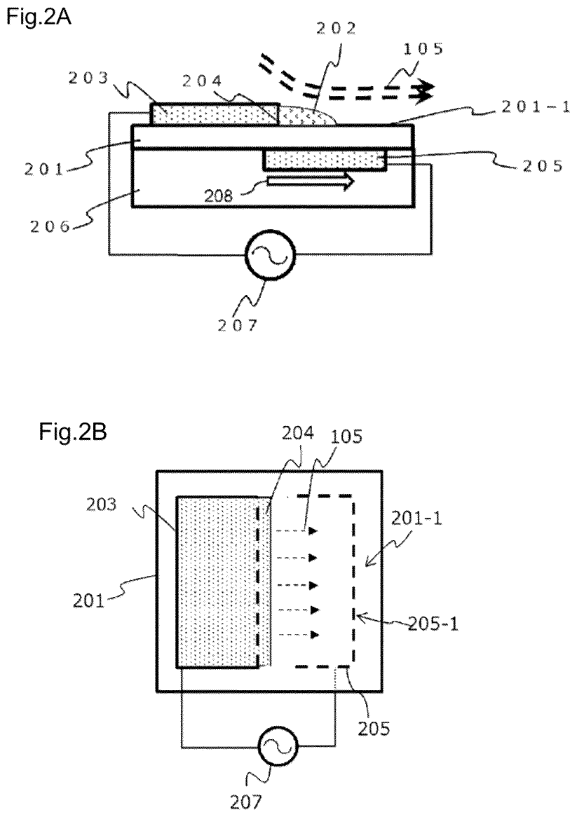

In addition, FIGS. 2A and 2B illustrate a cross-sectional structure of an embodiment of the plasma actuator 103. The plasma actuator is a so-called DBD (Dielectric Barrier Discharge) plasma actuator (which may be hereinafter referred to simply as the “DBD-PA”) in which, on one surface (hereinafter referred to also as the “first surface”) of a dielectric material 201, a exposed electrode (hereinafter referred to also as the “first electrode”) 203 having an exposed end surface, is provided while, on a surface (hereinafter referred to also as the “second surface”) opposite to the first surface, a second electrode 205 is provided. In FIG. 2A, a reference sign 206 denotes a dielectric substrate in which the second electrode 205 is to be embedded in a direction of a thickness of the plasma actuator so as to prevent induced flows from an end surface of the second electrode from being formed. Between the first electrode and the second electrode, a voltage can be applied by a power source 207.

In the plasma actuator 103, the first electrode 203 and the second electrode 205, which are arranged with the dielectric material 201 being interposed therebetween, for example, arranged staggered so as to diagonally oppose each other. By applying a voltage from the power supply 207 to between the electrodes (between both electrodes), a dielectric-barrier discharge from the first electrode 203 toward the second electrode 205 is generated. In addition, a plasma 202 is generated along an exposed portion (a portion not covered by the first electrode) 201-1 of a first surface of the dielectric material 201 from an edge 204 of the first electrode 203 in a direction (an arrow 208 in FIG. 2A) in which the second electrode extends.

Furthermore, at the same time, an air intake flow directed from a space inside a container toward an electrode is also generated. An electron in the surface plasma 202 collides with an airborne oxygen molecule, dissociates the oxygen molecule, and generates an oxygen atom. The resulting oxygen atom collides with an undissociated oxygen molecule and generates ozone. Therefore, due to actions of a jet-stream flow due to the surface plasma 202 and the air intake flow, the induced flow 105 containing ozone is generated along a surface of the dielectric material 201 from the edge 204 of the first electrode 203 toward an edge 205-1 of the second electrode 205 or, in other words, oriented in a first direction indicated by the arrow 208.

In addition, the plasma actuator 103 and the ozone decomposing device 102 are arranged so that the induced flow 105 flows out of the housing 107 from the opening portion 106 and is supplied to a treatment surface 104-1 of the object to be treated 104.

In other words, in the plasma actuator, the first electrode 203, the dielectric material 201, and the second electrode 205 are stacked in this order, and the first electrode 203 is the exposed electrode provided on the first surface of the dielectric material 201. When a voltage is applied between the first electrode 203 and the second electrode 205, the plasma actuator generates the dielectric barrier discharge directed from the first electrode 203 to the second electrode 205 and cause the induced flows to be blown out from the first electrode 203 in the first direction (direction of the arrow 208 in FIG. 2A), which is the one direction along the first surface of the dielectric material 201.

More specifically, the dielectric barrier discharge directed from the one edge portion 204 of the first electrode 203 to the second electrode 205 is caused, and the induced flows 105, which are unidirectional jet streams, are blown out from the one edge portion 204 of the first electrode 203 in the first direction (direction of the arrow 208 in FIG. 2A) along the first surface of the dielectric material 201.

Meanwhile, in a cross section along a thickness direction of the plasma actuator, the second electrode 205 is present to extend in the direction (first direction) in which the induced flows are blown out. Therefore, directionality of the induced flow 105 in the first direction can be further enhanced.

More specifically, for example, the plasma actuator has the dielectric material 201 and, when the cross section along the thickness direction of the plasma actuator is viewed, the first electrode 203 and the second electrode 205 are arranged to obliquely face each other via the dielectric material 201 in the thickness direction of the plasma actuator. The first electrode 203 is provided so as to cover a portion of the first surface of the dielectric material 201, and the first surface of the dielectric material has the exposed portion 201-1 uncovered with the first electrode 203.

FIG. 2B is a diagram when the plasma actuator is viewed through from a side of one surface of the dielectric material. At least a part of the exposed portion 201-1 and the second electrode 205 indicated by a dashed line overlap with each other. Therefore, the overlap is a region formed by an upper side, a lower side, and a right side of the dashed line indicating the electrode 205 and the edge 204 in FIG. 2B.

Furthermore, by applying a voltage between the first electrode and the second electrode, an induced flow containing ozone is generated along the exposed portion of the dielectric material which overlaps with the second electrode 205 from the edge 204 on a side of the first direction of the first electrode 203 in the cross section (FIG. 2A) in the thickness direction. Note that the first direction 208 is a direction from the edge 204 of the first electrode 203 toward the edge of the dielectric material and refers to a direction along the exposed portion 201-1 of the first surface of the dielectric material 201. In addition, normally, the exposed portion 201-1 of the first surface of the dielectric material 201 is a flat surface. Furthermore, the dielectric material 201 is preferably a rectangular sheet.

The induced flow is, for example, a wall-surface jet flow along the exposed portion 201-1 and readily supplies high-concentration ozone to a specific position. While a length in a blowout direction in a induced flow direction of the exposed portion 201-1 of the dielectric material 201 (in other words, a length from the edge 204 on a side of the first direction of the first electrode to the edge 205-1 of the second electrode on the first surface of the dielectric material) is not particularly limited, the length preferably ranges from 0.1 to 50 mm, more preferably ranges from 0.5 to 20 mm, and even more preferably ranges from 1 to 10 mm. The longer the length of the exposed portion 201-1 in the induced flow direction, the longer the plasma 202 stretches and the farther the induced flow reaches. On the other hand, when the length of the exposed portion 201-1 in the induced flow direction is too long, a distance to the opening portion 106 increases. Therefore, the ranges described above are preferable.

As the ozone decomposing device 102, the ultraviolet light source 102 irradiates the induced flow 105 with ultraviolet rays, decomposes ozone in the induced flow 105, and generates reactive oxygen inside the induced flow. In addition, as shown in FIGS. 1A and 1B, the plasma actuator 103 and the ultraviolet light source 102 are arranged so that the induced flow 105 containing reactive oxygen flows out of the housing 107 from the opening portion 106 and is supplied to the treatment surface 104-1 of the object to be treated 104.

Note that in FIG. 1A, a surface of the object to be treated 104 is also irradiated with the ultraviolet rays from the ultraviolet light source 102. In this case, even if all of the ozone in the induced flow 105 is not decomposed to reactive oxygen in the reactive oxygen supply device, since ozone having reached the surface of the object to be treated 104 is decomposed in situ by ultraviolet rays and becomes reactive oxygen, an improvement in treatment efficiency is expected.

However, in the reactive oxygen supply device according to the present disclosure, irradiation of the object to be treated by ultraviolet rays from the ultraviolet light source is not an essential configuration. For example, a plasma actuator configured such that the ultraviolet light source 102 is not directly visible from the opening portion 106 as shown in FIG. 1B is also within a range of the present disclosure. In the plasma actuator shown in FIG. 1B, as a result of ozone decomposed due to ultraviolet rays from the ultraviolet light source 102, the induced flow 105 containing reactive oxygen flows out from the opening portion 106 and is supplied to the treatment surface 104-1 of the object to be treated 104.

In other words, in the treatment apparatus according to an aspect of the present disclosure, the ozone in the induced flow 105 containing ozone from the plasma actuator (plasma generating apparatus) 103 of the reactive oxygen supply device 101 is decomposed by the ozone decomposing device 102. Specifically, for example, due to the ultraviolet light source 102 irradiating the induced flow 105 with ultraviolet rays, the induced flow 105 becomes a flow containing reactive oxygen. Furthermore, the induced flow 105 containing reactive oxygen flows out of the housing 107 from the opening portion 106 and is supplied to the treatment surface 104-1 of the object to be treated 104. As a result, reactive oxygen can be actively supplied to a region in a vicinity of the treatment surface 104-1 of the object to be treated 104 or, specifically, for example, a spatial region (hereinafter, also referred to as a “surface region”) from the treatment surface 104-1 to a height of around 1 mm.

Therefore, the generated reactive oxygen can be supplied to the surface of the object to be treated before the reactive oxygen is converted into oxygen and water. As a result, the treatment surface 104-1 of the object to be treated 104 is more readily treated by reactive oxygen.

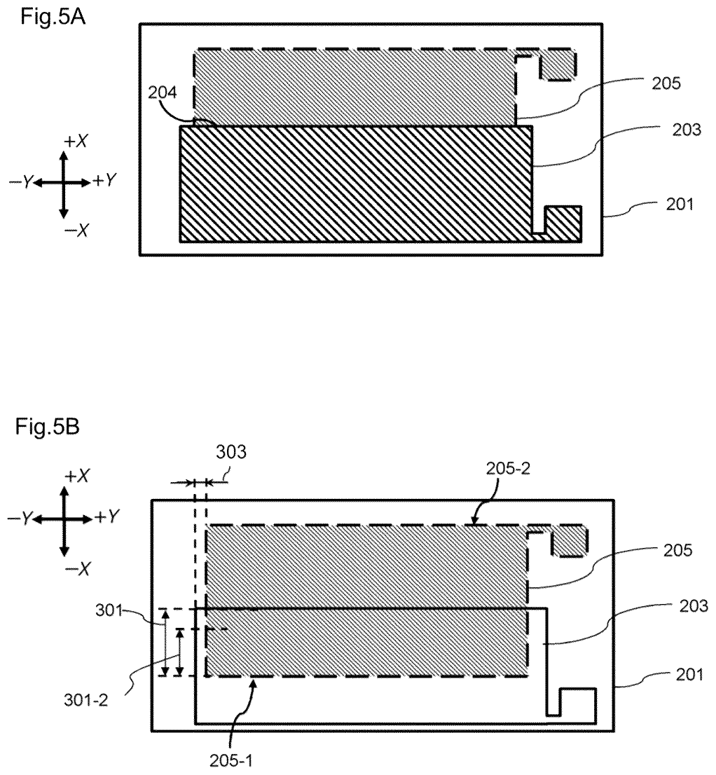

FIG. 5A illustrates a plan view resulting from observation from the first electrode 203 side when it is assumed that the dielectric material 201 of the plasma actuator 103 is transparent. In FIG. 5A, an X-axis is an axis parallel to the direction (first direction) in which the induced flows 105 from the plasma actuator 103 are blown out, and the first direction is a +X-direction. Meanwhile, a Y-axis is an axis perpendicular to the X-axis and, in FIG. 5A, a direction extending to a right side is a +Y-direction, while a direction extending to a left side is a −Y-direction.

The first electrode 203 is provided on the first surface of the dielectric material 201 to cover a portion of the surface of the dielectric material 201.

Alternatively, the plasma actuator may also be a so-called three-electrode plasma actuator in which a third electrode is further provided downstream in the direction in which the induced flows are blown out from the first electrode and on the first surface of the dielectric material 201. In this case, for example, it is possible to apply an ac voltage by using the first electrode as an AC electrode and apply a dc voltage by using the third electrode as a DC electrode. By applying a negative DC voltage to the DC electrode, it is also possible to generate a sliding discharge.

FIG. 5B is a perspective view obtained by viewing the plasma actuator 103 from the first electrode 203 side thereof. As illustrated in FIG. 5B, an edge portion 205-1 of the second electrode 205 leading in the −X-direction, which is embedded in the thickness direction of the plasma actuator 103, is located in the −X-direction to be ahead of a most leading position in the +X-direction of the edge portion 204 of the first electrode 203 leading in the +X-direction. In other words, the first electrode 203 and the second electrode 205 overlap each other by a length 301 in an X-axis direction. It can also be said that, in other words, as illustrated in FIG. 2A which is a cross-sectional view of the plasma actuator 103 along the thickness direction thereof, the first electrode 203 and the second electrode 205 are arranged to obliquely face each other with the dielectric material being interposed therebetween in the thickness direction of the plasma actuator 103.

Note that the edge 204 on the side of the first direction of the first electrode is considered an edge A and the edge 205-1 on the side of the second direction (side of −X direction) that is an opposite direction to the first direction in the second electrode is considered an edge B. The overlap length 301 of a position closest to the first direction (closest to the +X direction) of the edge A and the edge B may be hereinafter referred to as an “overlap amount”.

The shorter a shortest distance between the first electrode and the second electrode, the more readily plasma will occur. Therefore, a thickness of a portion of the dielectric material 201 that is present between the first electrode 203 and the second electrode 205 is preferably made thin within a range in which breakdown does not occur when a voltage is applied to both electrodes. Specifically, for example, when the applied voltage is to range from AC 100 V to AC 10000 V, the thickness of the dielectric portion can be set from 10 μm to 1000 μm and preferably from 10 μm to 200 μm. In addition, the shortest distance between the first electrode and the second electrode is preferably 200 μm or less.

An edge 205-2 on the +X direction side of the second electrode 205 is preferably closer to the +X direction than the edge 204 in the +X direction of the first electrode 203. Due to the second electrode extending farther in the +X direction than the edge 204 of the first electrode 203, directionality of the induced flow 105 in the +X direction can be further enhanced.

In addition, as shown in FIGS. 5A and 5B, the second electrode 205 is provided so as to overlap with the first electrode while sandwiching the dielectric material and extends in the +X direction. By applying a voltage between the first electrode 203 and the second electrode 205 configured in this manner, a stronger induced flow 105 can be created in the +X direction from the edge 204 of the first electrode of the plasma actuator 103.

Irradiating such an induced flow with ultraviolet rays from the ultraviolet light source causes ozone in the induced flow to be decomposed by reactive oxygen and the induced flow is to contain the reactive oxygen. According to an examination carried out by the present inventors, the reactive oxygen contained in such an induced flow can conceivably maintain its reactive state longer than generally-considered lifespans of reactive oxygen (half-life of O₂⁻: 10⁻⁶ seconds, half-life of OH: 10⁻⁹ seconds). This is conceivably due to the fact that the reactive oxygen in the induced flow is protected in the regulated flow of the induced flow, collisions with other reactive species or airborne molecules are suppressed, and deactivation caused by reactions is less likely to occur.

As a result, with the reactive oxygen supply device according to the present disclosure, reactive oxygen can be caused to reach the object to be treated more reliably. In other words, reactive oxygen can be more actively supplied to the object to be treated.

In addition, the conveying means 109 is capable of conveying the object to be treated in at least the direction A and conveys the object to be treated 104 in a direction of the arrow 108 so that the induced flow 105 containing reactive oxygen which flows outside of the housing 107 from the opening portion 106 is supplied to the treatment surface 104-1 of the object to be treated. In other words, the reactive oxygen supply device 101 and the conveying means 109 are arranged so that the induced flow 105 containing reactive oxygen which flows outside of the housing 107 from the opening portion 106 is supplied to the treatment surface 104-1 of the object to be treated which is conveyed in the direction of the arrow 108 by the conveying means 109.

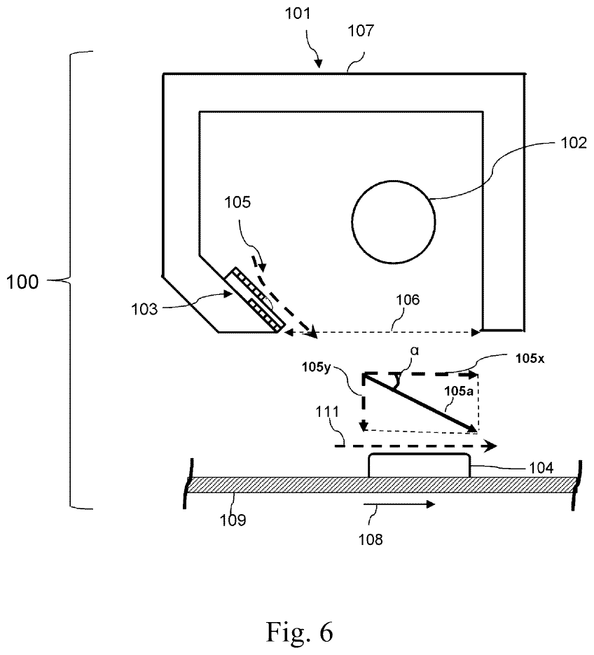

Furthermore, in the treatment apparatus 100, as shown in FIG. 6, an outflow direction vector 105a of the induced flow 105 containing reactive oxygen which flows outside of the housing 107 from the opening portion 106 includes a vector component 105x (hereinafter, also referred to as a vector component x) which is parallel to and oriented in a same direction as the direction of the arrow 108 being the conveying direction A of the object to be treated 104. In other words, the vector component x is a vector component in a forward direction of the direction A.

By setting the outflow direction of the induced flow 105 from the opening portion and the conveying direction of the object to be treated 104 so as to satisfy the relationship described above and arranging the reactive oxygen supply device 101 and the conveying means 109 as described above, a treatment effect by reactive oxygen of a moving object to be treated can be dramatically improved.

The present inventors infer the reason therefor as follows. As described above, in a regulated flow of the induced flow supplied from the reactive oxygen supply device according to the present disclosure, reactive oxygen can maintain a reactive state over a longer period of time. On the other hand, an air flow 111 around the object to be treated being conveyed in the direction of the arrow 108 naturally flows in the direction of the arrow 108. Therefore, due to the outflow direction vector 105a of the induced flow containing the component 105x that is parallel to the direction of the arrow 108 being the conveying direction A of the object to be treated 104, a regulated air flow of the induced flow 105 supplied to the object to be treated 104 is readily disturbed by a surrounding air flow created by the conveyance of the object to be treated 104 and, consequently, the object to be treated can be placed in an environment where reactive oxygen is present over a longer period of time. As a result, the treatment effect of the object to be treated by reactive oxygen conceivably increases.

The outflow direction vector is a vector in a direction from the edge 204 of the first electrode 203 toward a surface 201-1 of the dielectric material in the induced flow containing reactive oxygen which flows outside of the housing from the opening portion. In addition, normally, the outflow direction vector is a vector in a same direction as the first direction. The induced flow containing reactive oxygen which flows outside of the housing from the opening portion mainly flows out in the first direction and is supplied to the object to be treated that is conveyed in the direction A by the conveying means.

The outflow direction vector is determined by an installation angle of the plasma actuator 103.

The outflow direction vector 105a may be a vector solely constituted of the vector component x.

In addition, the outflow direction vector 105a may further include a vector component 105y (hereinafter, also referred to as a vector component y) in a direction from the opening portion 106 toward the treatment surface 104-1 of the object to be treated among vector components perpendicular to the direction A. A ratio of a magnitude of the vector component y with respect to a magnitude of the vector component x (vector component y/vector component x) preferably ranges from 0.00 to 2.75 and more preferably ranges from 0.58 to 2.75.

Furthermore, the outflow direction vector 105a may sometimes further include a vector component z that is a vector component perpendicular to the vector component x and the vector component y. A ratio of a magnitude of the vector component z with respect to a magnitude of the vector component x (vector component z/vector component x) preferably ranges from 0.00 to 2.75, more preferably ranges from 0.00 to 0.58, and even more preferably is 0.00. The vector component z that is perpendicular to the vector component x and the vector component y is either a vector component oriented in a depth direction in FIG. 6 or a vector component oriented in a vertical direction relative to a paper plane of FIG. 6.

When the ratio of a magnitude of the vector component y or the vector component z with respect to a magnitude of the vector component x is within the ranges described above, the induced flow 105 containing reactive oxygen can be more efficiently supplied to the treatment surface 104-1 of the object to be treated.

An angle α formed between the outflow direction vector and the direction A preferably ranges from 0° to 70° and more preferably ranges from 30° to 70°. When the formed angle α is within the ranges described above, an induced flow containing reactive oxygen is more readily supplied to the object to be treated. In FIG. 6, the formed angle α when the outflow direction vector is constituted of the vector components x and y is represented as an angle formed between the vector component 105x parallel to the direction of the arrow 108 that is the conveying direction A of the object to be treated 104 and the outflow direction vector.

The formed angle α of the outflow direction vector 105a can be measured as follows. As shown in FIGS. 2A and 2B, the induced flow 105 containing ozone is generated from an edge of the first electrode 203 in a direction of the electrode 205 arranged diagonally opposite to the first electrode 203 with the dielectric material 201 therebetween. Since the induced flow 105 proceeds in a direction along the dielectric material 201, the outflow direction vector 105a is a vector in a same direction as a vector in a direction toward the surface 201-1 of the dielectric material from the edge 204 of the first electrode 203.

Therefore, as a measurement method of the formed angle α of the outflow direction vector 105a, the formed angle α may be measured by a method of photographing the installation angle of the plasma actuator 103 from the z direction (not illustrated) using an apparatus for measuring dimensions such as a three-dimensional measuring machine or an image photographing apparatus such as a digital camera, a CCD camera, or a high-speed camera and measuring an angle formed by the surface 201-1 of the dielectric material of the plasma actuator 103 relative to the x axis or a method of directly measuring the formed angle α by a protractor or a digital angle meter.

An outflow speed of the induced flow 105 containing reactive oxygen which flows outside of the housing 107 from the opening portion 106 toward the outflow direction vector 105a preferably ranges from 1 m/s to 100 m/s and more preferably ranges from 10 m/s to 100 m/s. In addition, a movement speed in the direction A of the object to be treated that is conveyed by the conveying means 109 preferably ranges from 0.001 m/s to 5.000 m/s and more preferably ranges from 0.001 m/s to 1.000 m/s.

Furthermore, a ratio of the movement speed to the outflow speed preferably ranges from 0.001 to 1.000 and more preferably ranges from 0.001 to 0.100.

When the outflow speed and the movement speed are within the ranges described above or when the ratio of the movement speed to the outflow speed is within the ranges described above, a regulated air flow of the induced flow supplied from the reactive oxygen supply device is less readily disturbed and reactive oxygen can be more efficiently supplied to the object to be treated.

For example, the outflow speed can be adjusted by a blowout speed of the induced flow 105 from the plasma actuator 103 (hereinafter, also referred to as a flow rate of the induced flow) in the blowout direction (first direction) of the induced flow 105 containing ozone.

Materials that constitute the first electrode and the second electrode are not particularly limited as long as the materials exhibit good conductivity. For example, a metal such as copper, aluminum, stainless steel, gold, silver, or platinum, such a metal subjected to plating or vapor deposition, a conductive carbon material such as carbon black, graphite, or a carbon nanotube, or a composite material created by mixing such materials with a resin can be used. The material constituting the first electrode and the material constituting the second electrode may be the same or may differ from each other.

Among these materials, the material constituting the first electrode is preferably aluminum, stainless steel, or silver from the perspective of avoiding corrosion of the electrode and homogenizing discharge. For the same reasons, the material constituting the second electrode is also preferably aluminum, stainless steel, or silver.

In addition, as shapes of the first electrode and the second electrode, a flat plate shape, a wire shape, a needle shape, and the like can be adopted without particular restriction. Preferably, the shape of the first electrode is a flat plate shape. In addition, the shape of the second electrode is a flat plate shape. When at least one of the first electrode and the second electrode has a flat plate shape, an aspect ratio (length of long side/length of short side) of the flat plate is preferably 2 or more.

A material of the dielectric material is not particularly limited as long as the material has a high electrical insulation property. For example, a resin such as polyimide, polyester, a fluorine resin, a silicone resin, an acrylic resin, or a phenolic resin, glass, ceramics, or a composite material mixing glass or ceramics with a resin can be used. Among such materials, the dielectric material is preferably made of ceramics or glass since fire is unlikely to spread to ceramics or glass in case of a current leakage.

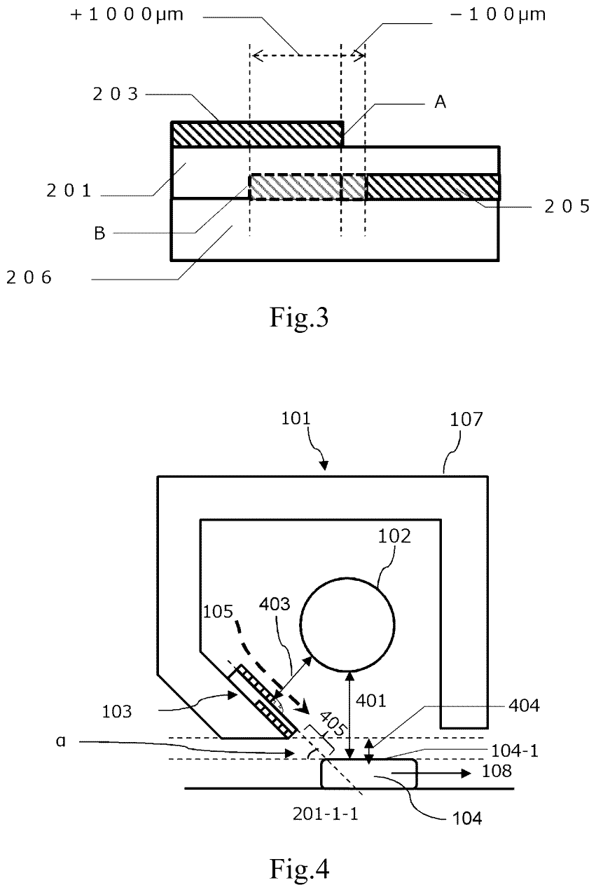

When an overlapping length is considered positive, an overlap of the edge A of the first electrode and the edge B of the second electrode in the X axis direction as viewed from above the sectional view preferably ranges from −100 μm to +1000 μm, more preferably ranges from 0 μm to +200 μm, and is even more preferably 0 μm (FIG. 3). In other words, when a case where the edge B is positioned more toward an opposite side to the blowout direction of the induced flow than the edge A is considered positive, an interval between the edge A and the edge B in a direction along the surface of the dielectric material (in the X axis direction) preferably ranges from −100 μm to +1000 μm, more preferably ranges from 0 μm to +200 μm, and is even more preferably 0 μm.

While thicknesses of the first electrode and the second electrode are both not particularly limited, the thicknesses can be set so as to range from 10 μm to 1000 μm. When 10 μm or more, resistance decreases and plasma is more readily generated. When 1000 μm or less, since electric field concentration occurs more readily, plasma is more readily generated.

While widths of the first electrode and the second electrode are both not particularly limited, the widths can be set to 1000 μm or more.

In addition, when the edge of the second electrode is exposed, plasma is also generated from the edge of the second electrode and an induced flow oriented toward an opposite side to the induced flow 105 originating from the first electrode can be created. In the reactive oxygen supply device according to the present aspect, an ozone concentration of an internal space of the reactive oxygen supply device other than a surface region of the object to be treated is preferably minimized. Furthermore, preferably, a flow of gas that disturbs a flow of the induced flow 105 is not generated in the container. Therefore, an induced flow originating from the second electrode is preferably not generated. In consideration thereof, preferably, the second electrode 205 is covered by a dielectric material similar to a dielectric substrate 206 or embedded in the dielectric material 201 to prevent generation of plasma from the edge of the second electrode as shown in FIG. 2A and FIGS. 5A and 5B.

The second electrode need only be embedded to a degree that enables the generation of plasma from the edge of the second electrode to be prevented and, for example, a part of a surface of the second electrode may be exposed and the exposed surface of the second electrode and the dielectric substrate 206 or the dielectric material 201 may form a same plane. The edge of the second electrode is preferably covered by the dielectric substrate 206 or the dielectric material 201. Therefore, for example, the plasma actuator is preferably an SDBD (single dielectric barrier discharge) plasma actuator.

The induced flow 105 containing high-concentration ozone flows in a flow direction of a jet-like flow due to surface plasma along the exposed portion 201-1 of the first surface of the dielectric material 201 from the edge 204 of the first electrode 203 or, in other words, a direction from the edge 204 of the first electrode 203 along the exposed portion 201-1 of the first surface of the dielectric material. The induced flow is a flow of gas containing high-concentration ozone with a speed ranging from around several m/s to several ten m/s.

The voltage applied between the first electrode 203 and the second electrode 205 of the plasma actuator is not particularly limited as long as the voltage enables the plasma actuator to generate plasma. In addition, while the voltage may be either a DC voltage or an AC voltage, an AC voltage is preferable. Furthermore, making the voltage a pulse voltage is also a preferable aspect.

In addition, an amplitude and a frequency of the voltage can be appropriate set in order to adjust the flow rate of the induced flow and the ozone concentration in the induced flow. In this case, the amplitude and the frequency may be appropriately selected from the perspective of generating an ozone concentration necessary for generating an effective reactive oxygen concentration or an effective reactive oxygen amount in accordance with a purpose of treatment in the induced flow, supplying the generated reactive oxygen to a surface region of the object to be treated in a state where the effective reactive oxygen concentration or an effective reactive oxygen amount in accordance with a purpose of treatment is maintained, or the like.

For example, the amplitude of the voltage can be set so as to range from 1 kV to 100 kV. Furthermore, the frequency of the voltage is preferably 1 kHz or higher and more preferably ranges from 10 kHz to 100 kHz.

When an AC voltage is used as the voltage, while a waveform of the AC voltage is not particularly limited and a sine wave, a rectangular wave, a triangular wave, or the like can be adopted, a rectangular wave is preferably adopted from the perspective of a quick rise of voltage.

While a duty ratio of the voltage can also be appropriately selected, the rise of the voltage is preferably quick. Preferably, the voltage is applied so that the rise of voltage from a bottom to a peak of amplitude of a wavelength is 10,000,000 V/second or higher.

Note that a value obtained by dividing the amplitude of the voltage applied between the first electrode 203 and the second electrode 205 by a film thickness of the dielectric material 201 (voltage/film thickness) is preferably 10 kV/mm or more.

### <Ozone Decomposing Device>

The reactive oxygen supply device includes the ozone decomposing device 102. The ozone decomposing device decomposes ozone contained in an induced flow and generates reactive oxygen in the induced flow. Examples of the ozone decomposing device include an ozone decomposing device which is capable of acting on ozone contained in the induced flow and decomposing the ozone. The ozone decomposing device is preferably capable of decomposing ozone without disturbing a flow of the induced flow.

The ozone decomposing device is preferably at least one apparatus selected from a group consisting of an ultraviolet light source which irradiates an induced flow with ultraviolet rays and which generates reactive oxygen in the induced flow, a heating apparatus which heats an induced flow and which generates reactive oxygen in the induced flow, and a humidifying apparatus which humidifies an induced flow and which generates reactive oxygen in the induced flow. The ozone decomposing device may be a combination of these apparatuses. For example, the ozone decomposing device may be an apparatus which heats an induced flow while irradiating the induced flow with ultraviolet rays or an apparatus which humidifies the inside of the housing while irradiating an induced flow with ultraviolet rays and heating the induced flow. More preferably, the ozone decomposing device is an ultraviolet light source. Hereinafter, each apparatus will be described.

### <UV Light Source and UV Light>

The UV light source is not particularly limited as long as the UV light source can emit UV light that can excite the ozone and generate reactive oxygen. The UV light source is also not particularly limited as long as the UV light source has a wavelength and an illuminance of the UV light which are required to excite the ozone and obtain the effective reactive oxygen concentration or effective reactive oxygen amount according to the object of the treatment.

For example, a light absorption spectrum of the ozone has a peak value of 260 nm, and accordingly a peak wavelength of the UV light is preferably 220 nm to 310 nm, more preferably 253 nm to 285 nm, or still more preferably 253 nm to 266 nm.

As a specific UV light source, a low-pressure mercury lamp in which mercury is encapsulated together with an inert gas, such as argon or neon, in quartz glass, a cold cathode tube UV lamp (UV-CCL), a UV LED, or the like can be used. Wavelengths of the low-pressure mercury lamp and the cold cathode tube UV lamp may appropriately be selected from among 254 nm and the like. Meanwhile, from a viewpoint of output performance, a wavelength of the UV LED may appropriately be selected from among 265 nm, 275 nm, 280 nm, and the like.

### <Heating Apparatus>

The heating apparatus 102 is not particularly limited as long as the heating apparatus 102 is capable of exciting ozone in an induced flow and imparting enough thermal energy to generate reactive oxygen. Since thermal decomposition of ozone starts around 100° C., an apparatus capable of heating the induced flow to 120° C. is preferable. On the other hand, since thermal degradation such as fusion or decomposition may occur at over 120° C. depending on the object to be treated, a temperature of 200° C. or lower is preferable.

The heating apparatus is not particularly limited and examples of the heating apparatus include an apparatus equipped with a heat source (heat supplying means) which supplies heat. Specific examples include a ceramic heater, a cartridge heater, a sheathed heater, an electric heater, and an oil heater. In a case of an apparatus including a metal-based heating element, the heating element is preferably made of a material with superior oxidation resistance such as a nichrome-based alloy or tungsten. Preferably, a cartridge heater is used.

### <Humidifying Apparatus>

The humidifying apparatus 102 is not particularly limited as long as the humidifying apparatus 102 is capable of humidifying the inside of the housing to cause an induced flow to contain water and generating reactive oxygen in the induced flow by decomposing ozone in the induced flow using the water. In this case, humidifying refers to providing an object with moisture and a mode of the moisture is not particularly limited and may be at least one selected from a group consisting of gas, liquid, and solid. In addition, known water can be optionally used as the water used when providing moisture and the water may contain substances other than water.

The humidifying apparatus is not particularly limited and examples of the humidifying apparatus include a vaporizing humidifying apparatus and a mist-type humidifying apparatus.

In order to prevent humidity in a vicinity of the plasma actuator from rising, the humidifying apparatus preferably has directionality with respect to a direction in which moisture is supplied (hereinafter, also simply referred to as directionality). Due to the humidifying apparatus having directionality, a vicinity of an induced flow and a vicinity of a surface of the object to be treated can be efficiently humidified without humidifying a vicinity of the plasma actuator.

A known method can be suitably used to impart directionality to the humidifying apparatus. Examples of the method include a method of generating an air flow by providing a fan and transferring moisture in a direction of the air flow and a method of imparting moderate pressure to moisture by an air pump or the like and injecting the moisture in a desired direction. The directionality is preferably imparted in a same direction as an orientation of the induced flow (first direction) in order to avoid disturbing the flow of the induced flow.

### <Arrangement of Plasma Actuator, Ozone Decomposing Device, Conveying Means, and Object to be Treated>

In the reactive oxygen supply device 101, a position of the plasma actuator 103 which generates an induced flow containing ozone is not particularly limited as long as the plasma actuator 103 is arranged so that, due to ultraviolet rays emitted from the ultraviolet light source 102 that is an ozone decomposing device, the induced flow 105 flows out of the housing from the opening portion in a state where an effective reactive oxygen concentration or an effective reactive oxygen amount in accordance with a purpose of treatment is maintained and the induced flow 105 is supplied to the surface of the object to be treated. A similar description applies when the ozone decomposing device is a heating apparatus or a humidifying apparatus.

For example, the plasma actuator and the ozone decomposing device may be arranged so that the induced flow 105 containing the generated reactive oxygen is supplied to the surface of the object to be treated over a shortest distance.

In addition, for example, the plasma actuator may be arranged so that the treatment surface 104-1 of the object to be treated is included on an extension in a direction along (an exposed portion 201-1 of) the first surface of the dielectric material from the edge on the first direction side of the first electrode 203 of the plasma actuator. For example, the extension is preferably in tangent to the treatment surface 104-1.

In addition, an extension in a direction along the first surface of the dielectric material (same as +X direction) from an edge on the first direction side of the first electrode 203 of the plasma actuator preferably faces the opening portion. Accordingly, an induced flow can be readily caused to flow out of the housing from the opening portion.

Furthermore, in a case where the opening portion of the reactive oxygen supply device is oriented vertically downward, a narrow angle formed between an extension 201-1-1 of the direction along the exposed portion 201-1 of the first surface of the dielectric material from the edge of the first electrode of the plasma actuator and a horizontal plane (a plane perpendicular to a vertical direction) is denoted by a (hereinafter, also referred to as a plasma actuator angle of incidence or a PA angle of incidence; refer to FIG. 4). While the narrow angle α is not particularly limited as long as the narrow angle α is an angle which enables an induced flow to be actively supplied to a surface region of the object to be treated in a state where effective reactive oxygen or an effective reactive oxygen amount in accordance with a purpose of treatment is maintained or an angle that enables treatment of the surface region of the object to be performed by reactive oxygen, the narrow angle α is preferably more than 0° and 70° or less and more preferably ranges from 30° to 70°.

Arranging the plasma actuator and the ozone decomposing device as described above enables an induced flow containing reactive oxygen with a certain flow rate to be locally supplied to a region in a vicinity of the surface of the object to be treated or enables the region in a vicinity of the surface of the object to be treated to be treated by reactive oxygen. In addition, the induced flow having flowed out from the opening flows along the surface of the object to be treated and portions other than an opposed portion of the opening portion on a surface to be treated of the object to be treated are exposed to the induced flow containing reactive oxygen. Accordingly, a wider range of the surface to be treated 104-1 can be treated by reactive oxygen.

In addition, the plasma actuator may be arranged so that the treatment surface 104-1 of the object to be treated is included on an extension in the first direction (a blowout direction of the induced flow).

As long as the ozone decomposing device is arranged so as to generate reactive oxygen in the induced flow and treatment can be performed on a surface of the object to be treated in a state where an effective reactive oxygen concentration or an effective reactive oxygen amount in accordance with a purpose of treatment is maintained, the ozone decomposing device is otherwise not particularly limited.

As described above, an induced flow containing ozone is actively supplied to a region in a vicinity of the surface of the object to be treated. In addition, when the ozone decomposing device is an ultraviolet light source, reactive oxygen can be generated in the induced flow by irradiating the induced flow with ultraviolet rays. Therefore, by irradiating the induced flow with ultraviolet rays, ozone is excited, the induced flow in a state where reactive oxygen has been generated can be actively supplied to the surface of the object to be treated, and a reactive oxygen concentration or a reactive oxygen amount of the surface of the object to be treated can be significantly increased.

As long as the ozone decomposing device and the plasma actuator are respectively arranged so as to generate reactive oxygen in the induced flow and enable treatment to be performed on a surface of the object to be treated in a state where an effective reactive oxygen concentration or an effective reactive oxygen amount in accordance with a purpose of treatment is maintained, relative positions of the ozone decomposing device and the plasma actuator are otherwise not particularly limited.

In addition, since a distance between the ozone decomposing device and the plasma actuator also changes depending on the purpose of treatment, the distance cannot be flatly regulated. For example, a distance from the dielectric material of the plasma actuator to a surface opposing the ozone decomposing device is preferably set to 10 mm or less and more preferably set to 4 mm or less. However, the plasma actuator need not be placed at a location within around 10 mm from the ozone decomposing device and the distance between the ozone decomposing device and the plasma actuator is not particularly limited as long as the reactive oxygen in the induced flow can be set to an effective concentration in accordance with a purpose of treatment in a relationship with elements that enable ozone to be decomposed such as an illuminance or a wavelength of ultraviolet rays to be described later.

Furthermore, providing at least one of the ozone decomposing device and the plasma actuator with moving means and making at least one of the ozone decomposing device and the plasma actuator movable so that degrees of ozonolysis become uniform is also a preferable aspect.

As relative positions of the reactive oxygen supply device and the conveying means, at least one of the reactive oxygen supply device and the conveying means may be arranged so that reactive oxygen is generated in the induced flow and a surface of the object to be treated that is conveyed by the conveying means is exposed to the induced flow of which an effective reactive oxygen concentration or an effective reactive oxygen amount in accordance with a purpose of treatment is maintained.

In addition, when the ozone decomposing device is an ultraviolet light source, the ultraviolet light source may be arranged at a position where the surface of the object to be treated can be irradiated with ultraviolet rays or a position where the surface of the object to be treated cannot be irradiated with ultraviolet rays. Even when the surface of the object to be treated cannot be irradiated with ultraviolet rays from the ultraviolet light source, the treatment apparatus using reactive oxygen according to the present aspect enables treatment to be performed by exposing the surface to be treated to reactive oxygen in an induced flow.

In a similar manner, when the ozone decomposing device is a heating apparatus, the heating apparatus may be arranged at a position where the surface of the object to be treated can be heated or a position where the surface of the object to be treated cannot be heated.

Furthermore, in sterilization treatment using the UV light, what is sterilized is only the surface irradiated with the UV light. However, in the sterilization treatment using the reactive oxygen supply device according to the present disclosure, bacteria present at a position that can be reached by the reactive oxygen can be sterilized. Accordingly, for example, even bacteria present between fibers, which is hard to sterilize with UV light irradiation from the outside, may be sterilized.

On the other hand, when the ultraviolet light source is arranged so that the surface of the object to be treated placed outside the housing can be irradiated with ultraviolet rays from the ultraviolet light source via the opening portion, undecomposed ozone present in the induced flow can be decomposed in situ on the surface to be treated and reactive oxygen can be generated on the surface to be treated. As a result, a degree of treatment or an efficiency of treatment can be further enhanced.

In this case, while an illuminance of ultraviolet rays on the surface of the object to be treated or an illuminance of ultraviolet rays at the opening portion is not particularly limited, for example, an illuminance of ultraviolet rays is preferably set which enables ozone contained in an induced flow to be decomposed, reactive oxygen to be generated in the induced flow, and an effective reactive oxygen concentration or an effective reactive oxygen amount in accordance with a purpose of treatment to be generated even on the surface of the object to be treated or at the opening portion. Specifically, for example, a specific example of the illuminance of ultraviolet rays on the surface of the object to be treated or the illuminance of ultraviolet rays at the opening portion is preferably 40 μW/cm² or more, more preferably 100 μW/cm² or more, even more preferably 400 μW/cm² or more, and particularly preferably 1000 μW/cm² or more. While an upper limit of the illuminance is not particularly limited, for example, the illuminance can be set to 10000 μW/cm² or less. A preferable range is, for example, from 40 μW/cm² to 10000 μW/cm².

Furthermore, although a distance between the ozone decomposing device and the surface of the object to be treated also changes depending on the purpose of treatment and cannot be flatly regulated, for example, the ozone decomposing device and the conveying means are preferably arranged so that the distance is 10 mm or less or more preferably 4 mm or less. However, the object to be treated need not be placed at a location within around 10 mm from the ozone decomposing device and the distance between the ozone decomposing device and the object to be treated is not particularly limited as long as the reactive oxygen in the induced flow can be set to an effective concentration in accordance with a purpose of treatment in a relationship with elements that enable ozone to be decomposed such as an illuminance of the ultraviolet rays.

In addition, an amount of ozone generation per unit time in the plasma actuator in a state where the ozone decomposing device is prevented from decomposing the ozone in the induced flow is, for example, preferably 8 μg/minute or more. More preferably, the amount of ozone generation is 15 μg/minute or more. While an upper limit of the amount of ozone generation is not particularly limited, for example, the amount of ozone generation can be set to 1000 μg/minute or less. A preferable range is, for example, from 8 μg/minute to 1000 μg/minute.

As a flow rate of the induced flow, for example, the flow rate need only be a speed which enables generated reactive oxygen to be actively supplied to a surface region of the object to be treated in a state where an effective reactive oxygen concentration or an effective reactive oxygen amount in accordance with a purpose of treatment is maintained. For example, the flow rate ranges from around 0.01 m/s to 100 m/s as described earlier.

As described above, the concentration of ozone in an induced flow generated from the plasma actuator and a flow rate of the induced flow can be controlled by thicknesses and materials of the electrodes and the dielectric material, a type, an amplitude, and a frequency of voltage to be applied, and the like.

### <Housing and Opening Portion>

The reactive oxygen supply device comprises the housing 107 including at least one opening portion 106, the ozone decomposing device 102 arranged inside the housing, and the plasma actuator 103.

The opening portion is not particularly limited as long as a mode of the opening portion causes the induced flow 105 generated from the plasma actuator 103 to flow outside the housing 107. A size of the opening portion, a position of the opening portion, and relative positions of the opening portion and the conveying means can be appropriately selected so that, for example, generated reactive oxygen can be actively supplied to a surface region of the object to be treated that is conveyed by the conveying means in a state where an effective reactive oxygen concentration or an effective reactive oxygen amount in accordance with a purpose of treatment is maintained.

Furthermore, a distance between the plasma actuator and the opening portion is preferably set so that a distance between the plasma actuator and the object to be treated is close enough in order to use the reactive oxygen in the induced flow for aimed treatment more effectively. Therefore, the plasma actuator is preferably arranged at a closer position to the opening portion. On the other hand, arranging the plasma actuator at a position set back from the opening portion is also preferable in order to protect the plasma actuator. As an example, the plasma actuator is preferably arranged on an inner wall of the housing so that an edge close to the opening portion of the plasma actuator is positioned 0.5 mm to 1.5 mm from an edge of the opening portion on the inner wall of the housing.

### <Conveying Means>

The treatment apparatus 100 according to the present disclosure comprises the conveying means 109 capable of conveying the object to be treated 104 in at least a direction of the arrow 108 (direction A). The conveying means 109 is not particularly limited as long as a mode of the conveying means 109 enables the object to be treated 104 to be conveyed and causes the induced flow 105 containing reactive oxygen which flows outside of the housing 107 to be supplied to the treatment surface 104-1 of the object to be treated. A shape of the conveying means, a conveying distance over which the conveying means conveys the object to be treated, and a speed at which the object to be treated is moved in the direction A can be appropriately selected so that, for example, generated reactive oxygen can be actively supplied to a surface region of the object to be treated in a state where an effective reactive oxygen concentration or an effective reactive oxygen amount in accordance with a purpose of treatment is maintained. Examples of specific conveying means include a conveyor, air conveyance, magnetic conveyance, and a multi-jointed robot.

The conveying means may be capable of conveying the object to be treated in a direction other than the direction A before and after the to be treated is treated by the induced flow containing reactive oxygen.

A distance over which the object to be treated is conveyed in the direction A by the conveying means can be appropriately selected depending on intended use and is not particularly limited as long as a mode of the conveying means causes the induced flow containing reactive oxygen to be supplied to the object to be treated that is conveyed in the direction A.

### <Object to be Treated>

A shape, a material, a size, and the like of the object to be treated can be appropriately selected depending on the intended use. At least a part of the surface of the object to be treated is preferably made of paper (such as filter paper), glass, ceramic, plastic, rubber, cloth, metal, or the like.

One object to be treated or a plurality of objects to be treated may be conveyed by the conveying means and, when there are a plurality of objects to be treated, the plurality of objects to be treated may be successively conveyed by the conveying means.

The reactive oxygen supply device in the present disclosure can be used not only for an application in which the object to be treated is sterilized, but also for all the applications that are implemented by supplying the reactive oxygen to the object to be treated. For example, the reactive oxygen supply device in the present disclosure can be used also for an application in which the object to be treated is deodorized, an application in which the object to be treated is bleached, hydrophilization surface treatment for the object to be treated, or the like.

Meanwhile, the device for performing treatment with reactive oxygen in the present disclosure can be used not only for treatment of sterilizing the object to be treated, but also for, e.g., treatment of deodorizing the object to be treated, treatment of bleaching the object to be treated, a surface treatment of hydrophilizing the object to be treated, or the like.

In addition, the present disclosure provides a treatment method of treating an object to be treated using reactive oxygen, comprising:
a step of preparing a reactive oxygen supply device and conveying means capable of conveying the object to be treated with reactive oxygen in at least a direction A, wherein
the reactive oxygen supply device comprises a plasma actuator and an ozone decomposing device inside a housing having at least one opening portion,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge from the first electrode to the second electrode and cause an induced flow containing ozone to be blown out from the first electrode in a first direction, which is one direction along the surface of the dielectric material,
the ozone decomposing device generates reactive oxygen in the induced flow by decomposing the ozone contained in the induced flow and the induced flow becomes an induced flow containing reactive oxygen,
the plasma actuator and the ozone decomposing device are arranged such that the induced flow containing the reactive oxygen flows out from the opening portion to the outside of the housing,
the treatment method further comprises a step of causing the induced flow containing the reactive oxygen to flow out from the opening portion and supplying the induced flow containing the reactive oxygen to the object to be treated, which is moved in the direction A, and
an outflow direction vector of the induced flow containing the reactive oxygen flowed out from the opening portion has a vector component x which is parallel to and oriented in the same direction as the direction A.

Note that, in the present disclosure, the “effective reactive oxygen concentration or effective reactive oxygen amount” refers to a reactive oxygen concentration or a reactive oxygen amount for attaining an object with respect to the object to be treated such as, e.g., sterilization, deodorization, bleaching, or hydrophilization, and can be adjusted appropriately according to the object by using the thicknesses and materials of the electrodes and the dielectric material which are included in the plasma actuator, the type, amplitude, and frequency of the voltage to be applied, a degree of ozone decomposition (the illuminance and irradiation time of the UV light, the heating temperature, the heating time, a humidification moisture amount, and a humidification time) by the ozone decomposing device, the PA incidence angle, or the like.

### EXAMPLES

Using Examples and Comparative Examples, the following will describe the present disclosure in greater detail, but the embodiments of the present disclosure are not limited thereto.

### First Example

### 1. Production of Reactive Oxygen Supply Device

A first electrode was formed by bonding a piece of aluminum foil with a length of 2.5 mm, a width of 15 mm, and a thickness of 100 μm using a piece of adhesive tape to a first surface of a glass plate (length: 5 mm, width (depth direction of paper plane in FIG. 2A): 18 mm, and thickness: 150 μm) as a dielectric material. In addition, a second electrode was similarly formed by bonding a piece of aluminum foil with a length of 3 mm, a width of 15 mm, and a thickness of 100 μm using a piece of adhesive tape to a second surface of the glass plate so as to diagonally oppose the piece of aluminum foil bonded to the first surface. Furthermore, the second surface including the second electrode was covered by a piece of polyimide tape. In this manner, a plasma actuator in which the first electrode and the second electrode were provided so as to overlap with each other by a width of 0.5 mm while sandwiching the dielectric material (glass plate) was fabricated. Two of the plasma actuators were prepared.

Next, as the housing 107 of the reactive oxygen supply device 101, a case made of an ABS resin with a height of 25 mm, a width of 20 mm, a length of 170 mm, a thickness of 2 mm, and a sectional shape being the approximately trapezoidal shape shown in FIG. 1A was prepared. The case included, on one face, the opening portion 106 with a width of 7 mm and a length of 166 mm. Next, the plasma actuator fabricated earlier was fixed to an inner wall of a hypotenuse portion of the housing 107. Specifically, the plasma actuator 103 was arranged so that an angle α formed by an intersection point of the extension 201-1-1 of a direction along the exposed portion 201-1 of the first surface of the dielectric material 201 and the treatment surface 104-1 of the object to be treated (a same value as the PA angle of incidence described earlier) was 45°.

Furthermore, the ultraviolet lamp 102 (a cold-cathode tube ultraviolet lamp, trade name: UW/9F89/9, manufactured by STANLEY ELECTRIC CO., LTD., a cylindrical shape with a diameter of 9 mm, peak wavelength=254 nm) was arranged inside the housing. The ultraviolet lamp 102 was arranged so that a shortest distance (reference sign 403 in FIG. 4) between the ultraviolet lamp 102 and the exposed portion 201-1 of the first surface of the dielectric material 201 of the plasma actuator was 2 mm and a distance (reference sign 401 in FIG. 4) between the ultraviolet light source and a surface on a side opposing the ultraviolet light source when a flat plate was abutted against the opening portion 106 of the housing 107 was 3 mm. The reactive oxygen supply device (treatment apparatus using reactive oxygen) according to the present example was fabricated in this manner.

An illuminance meter (trade name: Spectral radiometer USR-45D, manufactured by Ushio Inc.) was placed at a position of the opening portion 106 that acts as a supply port of reactive oxygen in the reactive oxygen supply device 101 and an illuminance of ultraviolet rays was measured. Based on a spectral integrated value, the illuminance was 1370 μW/cm². At this point, power of the plasma actuator was not turned on in order to avoid an effect of shielding ultraviolet rays by ozone generated from the plasma actuator. Since the object to be treated is placed at, for example, the position of the opening portion 106, the illuminance of ultraviolet rays measured under these conditions was considered the illuminance of ultraviolet rays on the surface of the object to be treated.

Next, in order to calculate an amount of ozone generated from the plasma actuator 103, the reactive oxygen supply device 101 was placed inside a sealable container (not illustrated) with a capacity of 1 liter. The sealable container was provided with a hole which can be sealed with a rubber plug and which enables inside air to be suctioned by a syringe from the hole. A 2.4-kVpp voltage with a frequency of 80 kHz and a sine waveform was applied to the plasma actuator 103 and 100 ml of the gas inside the sealable container was collected after 1 minute. The collected gas was suctioned by an ozone detector tube (trade name: 182SB, manufactured by KOMYO RIKAGAKU KOGYO K.K.) and a measured ozone concentration (PPM) contained in an induced flow from the plasma actuator 103 was measured. Using the measured value of ozone concentration, an ozone generation amount per unit time was obtained according to the following equation.

$$Amount of ozone generated per unit time ( mg / min ) = measured ozone concentration ( PPM ) * \begin{matrix}\begin{matrix}ozone molecular \\ weight ( 48 )\end{matrix} \\ 22.4\end{matrix} * \begin{matrix}\begin{matrix}273 \\ 273 + room temperature ( °C )\end{matrix} \\ 10000\end{matrix} * \frac{\begin{matrix}gas inside first \\ chamber ( L )\end{matrix}}{collected gas ( L )} = measured oxone concentration ( PPM ) * 48 / 22.4 * 273 / ( 273 + 25 ) / 10000 - 0.1 / 1 \qquad [ Math . 1 ]$$

As a result, the ozone generation amount per unit time was 19 μg/minute. At this point, power of the ultraviolet light source was not turned on in order to avoid an effect of ozonolysis due to the ultraviolet rays emitted from the ultraviolet light source.

Finally, an ozone generation amount when both the plasma actuator 103 and the ultraviolet lamp 102 were operational was measured. An operating condition of the plasma actuator 103 was a condition of generating 39 μg/minute of ozone when only operating the plasma actuator 103. In addition, an operating condition of the ultraviolet lamp 102 was a condition of achieving an illuminance of 1370 μW/cm² when only operating the ultraviolet lamp 102. As a result, the ozone generation amount when both the plasma actuator 103 and the ultraviolet lamp 102 were operational was 4 μg/minute. A decrease of 15 μg/minute from 19 μg/minute is conceivably an amount of ozone which had changed to reactive oxygen.

### 2-1. Detection Test of Reactive Oxygen (Absorbance of Methylene Blue)

The reactive oxygen supply device fabricated in 1 above was started up and a presence/absence of reactive oxygen in an induced flow flowing out from the opening of the housing was checked using a decoloration reaction of an aqueous solution of methylene blue. Specifically, methylene blue (manufactured by KANTO CHEMICAL CO., INC., special grade) and distilled water were mixed to prepare a 0.01% methylene blue aqueous solution. 15 ml of the methylene blue aqueous solution was placed in a petri dish (AB4000 manufactured by EIKEN CHEMICAL CO., LTD., columnar shape, diameter: 88 mm). In addition, assuming that a fluid surface of the methylene blue aqueous solution in the petri dish is the surface to be treated 104-1 of the object to be treated, the reactive oxygen supply device was arranged on the petri dish so that a distance 405 in FIG. 4 was 1.4 mm (a distance 404 was 1 mm).

Next, a 2.4-kVpp AC voltage with a frequency of 80 kHz and a sine waveform was applied between both electrodes of the plasma actuator of the reactive oxygen supply device, the ultraviolet lamp was lighted, and the induced flow having flowed out from the opening was supplied toward the fluid surface for 30 minutes. The ultraviolet lamp was adjusted so that an illuminance measured on an exposed surface on a side opposing the ultraviolet lamp of the dielectric material of the plasma actuator was 1370 μW/cm² without turning on the power of the plasma actuator.

The methylene blue aqueous solution after irradiation of the induced flow was transferred to a cell and a change in light absorption of methylene blue was measured using a spectrophotometer (V-570 manufactured by JASCO). Since methylene blue has a strong absorption at a wavelength of 664 nm, a degree of decoloration of methylene blue can be calculated from a change in absorbance of the wavelength. In the present test, only distilled water was first placed in a reference cell, and a measurement of the 0.01% methylene blue aqueous solution before irradiation of the induced flow having been placed in a sample cell resulted in an absorbance of 2.32 Abs. On the other hand, absorbance of the methylene blue aqueous solution after irradiation of the induced flow was 0.05 Abs. Therefore, a rate of decrease of absorbance was 88% ((2.32−0.27)/2.32)×100).

### 2-2. Treatment (Bacterial Eradication) Test

A bacterial eradication test of E. coli was performed using the reactive oxygen supply device 101 according to the procedure described below. Note that all tools and instruments used in the present bacterial eradication test were used after being subjected to autoclaving using an autoclave. In addition, the present bacterial eradication test was performed in a clean bench.

First, E. coli (trade name: “KWIK-STIK (Escherichia coli) ATCC8739)”, manufactured by Microbiologics, Inc.) was introduced to a conical flask holding an LB broth (a mixture of 2 g of tryptone (trade name: “Bacto Tryptone”, manufactured by Life Technologies Japan Ltd.), 1 g of a yeast extract (trade name: “Yeast Extract”, manufactured by Life Technologies Japan Ltd.), 1 g of sodium chloride (trade name: “sodium chloride special grade”, manufactured by KISHIDA CHEMICAL CO., LTD.), and 200 mL of distilled water). Next, the conical flask was cultured by shaking using a shaking incubator (TA-25R-3F, manufactured by Takasaki Scientific Instruments Corp.) at a temperature of 37° C. for 48 hours at 80 rpm and an E. coli solution was obtained. A viable count of the obtained E. coli solution was 9.2×10⁹ (CFU/mL).

0.010 mL of the bacterial solution after culture was dripped using a micropipette only onto one surface of a 3 cm by 1 cm qualitative filter paper (product number: No. 5C, manufactured by ADVANTEC CO., LTD.) to fabricate sample No. 1. Sample No. 2 was fabricated in a similar manner.

Next, sample No. 1 was immersed for 1 hour in a test tube holding 10 ml of a buffer solution (trade name: Gibco PBS, manufactured by Thermo Fisher Scientific Inc.). A period of time from dripping of the bacterial solution onto the filter paper to immersion in the buffer solution was set to 60 seconds in order to prevent drying of the bacterial solution on the filter paper.

Next, 1 ml of the buffer solution (hereinafter referred to also as the “1/1 solution”) after the immersion of Sample No. 1 therein was placed in a test tube containing 9 ml of the buffer solution to prepare a diluted solution (hereinafter referred to as the “1/10 diluted solution”). A 1/100 diluted solution, a 1/1000 diluted solution, and a 1/10000 diluted solution were prepared in the same manner, except that the dilution ratio with the buffer solution was changed.

Next, 0.050 ml was collected from a 1/1 solution and smeared on a stamp medium (PETAN CHECK 25PT1025, manufactured by EIKEN CHEMICAL CO., LTD.). This operation was repeated to prepare two stamp media smeared with the 1/1 solution. The two stamp media were placed in a constant temperature bath (trade name: IS600, manufactured by Yamato Scientific Co., Ltd.) and cultured at a temperature of 37° C. for 24 hours. The numbers of colonies generated on the two stamped media were counted and an average value thereof was calculated.

Two smeared stamp media were prepared and cultured for each of a 1/10 dilution, a 1/100 dilution, a 1/1000 dilution and a 1/10000 dilution in a similar manner to that described above. In addition, the numbers of colonies generated on the stamped media for each dilution were counted and an average value thereof was calculated. Results thereof are shown in Table 1.

TABLE 1

| | Sample No. 1 (blank) |
|---|---|
| 1/1 solution | >100 |
| 1/10 diluted solution | >100 |
| 1/100 diluted solution | 54 |
| 1/1000 diluted solution | 5 |
| 1/10000 diluted solution | 0 |

From the results in Table 1 above, it was found that the number of colonies was 54 when the 1/100 dilution was cultured and, therefore, the number of bacteria present in 0.050 ml of the 1/1 solution according to sample No. 1 was 54×10²=5400 (CFU).

Next, the following operations were performed with respect to sample No. 2.

Sample No. 2 as the object to be treated 104 was arranged so that the distance 404 in FIG. 4 between the bacterial solution-applied surface of a filter paper that is a surface to be treated and the reactive oxygen supply device 101 was 1 mm.

Next, a 2.4-kVpp AC voltage with a frequency of 80 kHz and a sine waveform was applied between both electrodes of the reactive oxygen supply device, the ultraviolet lamp was lighted, and an induced flow was supplied toward the filter paper. The ultraviolet lamp was adjusted so that an illuminance measured on an exposed surface on a side opposing the ultraviolet lamp of the plasma actuator was 1370 μW/cm² without turning on the power of the plasma actuator.

Subsequently, the reactive oxygen supply device was started up and the object to be treated 104 was treated while being conveyed at a speed of 0.050 m/s. A total of 10 passes of the treatment were performed, with one pass being a state where the object to be treated had completed passing through the opening portion 106 shown in FIG. 1A.

In addition, in the treatment process using the reactive oxygen supply device, a period of time from dripping of the bacterial solution onto the filter paper to immersion in the buffer solution was set to 60 seconds in order to prevent drying of the filter paper onto which the bacterial solution had been dripped.

Sample No. 2 after the treatment was immersed for 1 hour in a test tube holding 10 ml of a buffer solution (trade name: Gibco PBS, manufactured by Thermo Fisher Scientific Inc.) together with a piece of filter paper laid on a bottom of a depressed portion. Next, 1 ml of the buffer solution (hereinafter, a “1/1 solution”) after the immersion was introduced into a test tube containing 9 ml of a buffer solution to prepare a dilution (1/10 dilution). A 1/100 dilution, a 1/1000 dilution, and a 1/10000 dilution were prepared in a similar manner with the exception of changing a dilution factor by the buffer solution.

Next, 0.050 ml was collected from the 1/1 solution and smeared on a stamp medium (trade name: Petancheck 25PT1025, manufactured by EIKEN CHEMICAL CO., LTD.). This operation was repeated to prepare two stamp media smeared with the 1/1 solution. The total of two stamp media were placed in a constant temperature bath (trade name: IS600, manufactured by Yamato Scientific Co., Ltd.) and cultured at a temperature of 37° C. for 24 hours. The numbers of colonies generated on the stamped media with respect to the 1/1 solution were counted and an average value thereof was calculated. Two smeared stamp media were prepared and cultured for each of the 1/10 dilution, the 1/100 dilution, the 1/1000 dilution and the 1/10000 dilution in a similar manner to that described above. In addition, the numbers of colonies generated on the stamped media for each dilution were counted and an average value thereof was calculated. Results thereof are shown in Table 2.

TABLE 2

| | Sample No. 2 |
|---|---|
| 1/1 solution | 23 |
| 1/10 diluted solution | 2 |
| 1/100 diluted solution | 0 |
| 1/1000 diluted solution | 0 |
| 1/10000 diluted solution | 0 |

As shown in Table 2, compared to the number of bacteria in 0.050 ml of the 1/1 solution with respect to sample No. 1 which was not treated using the reactive oxygen supply device being 5400 (CFU), the number of bacteria in 0.050 ml of the 1/1 solution with respect to sample No. 2 after being treated was 23 (CFU). Accordingly, it was found that bacterial eradication of 99.57% ((5400−23/5400)×100) of sterilization was achieved by the 2-second treatment carried out by the reactive oxygen supply device according to the present example.

According to at least one aspect of the present disclosure, a treatment apparatus using reactive oxygen which is capable of more effectively treating a moving object to be treated with reactive oxygen can be obtained. In addition, according to another aspect of the present disclosure, a treatment method using reactive oxygen which is capable of more effectively treating a moving object to be treated with reactive oxygen can be obtained.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A treatment apparatus using reactive oxygen, comprising:
a reactive oxygen supply device and conveying means capable of conveying an object to be treated with reactive oxygen in at least a direction A, wherein
the reactive oxygen supply device comprises a plasma actuator and an ozone decomposing device inside a housing having at least one opening portion,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge from the first electrode to the second electrode and cause an induced flow containing ozone to be blown out from the first electrode in a first direction, which is one direction along the surface of the dielectric material,
the ozone decomposing device generates reactive oxygen in the induced flow by decomposing the ozone contained in the induced flow and the induced flow becomes an induced flow containing reactive oxygen,
the plasma actuator and the ozone decomposing device are arranged such that the induced flow containing the reactive oxygen flows out from the opening portion to the outside of the housing,
an outflow direction vector of the induced flow containing the reactive oxygen flowed out from the opening portion to the outside of the housing has a vector component x which is parallel to and oriented in the same direction as direction A, and
the reactive oxygen supply device and the conveying means are arranged such that the induced flow containing the reactive oxygen which flows out from the opening portion to the outside of the housing is supplied to a surface of the object to be treated which is conveyed by the conveying means.

2. The treatment apparatus using reactive oxygen according to claim 1, wherein
when a cross section of the plasma actuator along a thickness direction thereof is viewed,
the first electrode and the second electrode are arranged to obliquely face each other via the dielectric material in the thickness direction of the plasma actuator,
the first electrode is provided so as to cover a portion of the first surface of the dielectric material,
the first surface has an exposed portion uncovered with the first electrode,
when the plasma actuator is seen through from the first electrode side, at least a one portion of the exposed portion and the second electrode have overlap therebetween, and
the induced flow is blown out from an edge portion of the first electrode leading in the first direction in the cross section along the thickness direction along the exposed portion of the dielectric material overlapping the second electrode.

3. The treatment apparatus using reactive oxygen according to claim 1, wherein the outflow direction vector is a vector in a same direction as the first direction.

4. The treatment apparatus using reactive oxygen according to claim 1, wherein an angle α formed between the outflow direction vector and the direction A is from 0° to 70°.

5. The treatment apparatus using reactive oxygen according to claim 1, wherein
an outflow speed of the induced flow containing the reactive oxygen to the outflow direction vector is from 1 m/s to 100 m/s, and
a movement speed of the object to be treated which is conveyed by the conveying means in the direction A is from 0.001 m/s to 5.000 m/s.

6. The treatment apparatus using reactive oxygen according to claim 5, wherein a ratio of the movement speed to the outflow speed is from 0.001 to 1.000.

7. A treatment method of treating an object to be treated using reactive oxygen, comprising:
a step of preparing a reactive oxygen supply device and conveying means capable of conveying the object to be treated with reactive oxygen in at least a direction A, wherein
the reactive oxygen supply device comprises a plasma actuator and an ozone decomposing device inside a housing having at least one opening portion,
the plasma actuator comprises a first electrode, a dielectric material, and a second electrode which are stacked in this order,
the first electrode is an exposed electrode provided on a first surface, which is one surface of the dielectric material,
when a voltage is applied between the first electrode and the second electrode, the plasma actuator generates a dielectric barrier discharge from the first electrode to the second electrode and cause an induced flow containing ozone to be blown out from the first electrode in a first direction, which is one direction along the surface of the dielectric material,
the ozone decomposing device generates reactive oxygen in the induced flow by decomposing the ozone contained in the induced flow and the induced flow becomes an induced flow containing reactive oxygen,
the plasma actuator and the ozone decomposing device are arranged such that the induced flow containing the reactive oxygen flows out from the opening portion to the outside of the housing,
the treatment method further comprises a step of causing the induced flow containing the reactive oxygen to flow out from the opening portion and supplying the induced flow containing the reactive oxygen to the object to be treated, which is moved in the direction A, and
an outflow direction vector of the induced flow containing the reactive oxygen flowed out from the opening portion has a vector component x which is parallel to and oriented in the same direction as the direction A.
